(19) United States
(12) Patent Application Publication
(10) Pub. No.: US 2019/0156241 A1
(43) Pub. Date: May 23, 2019

(54) DATA ANALYSIS COLLABORATION ARCHITECTURE AND METHODS OF USE THEREOF

(71) Applicant: Calgary Scientific Inc., Calgary (CA)

(72) Inventor: Matthew Charles Hughes, Calgary (CA)

(21) Appl. No.: 16/192,998

(22) Filed: Nov. 16, 2018

Related U.S. Application Data
(60) Provisional application No. 62/587,762, filed on Nov. 17, 2017.
(60) Provisional application No. 62/590,515, filed on Nov. 24, 2017.

(51) Int. Cl. G06N 20/00; G06F 9/54; G06F 16/25; G06F 16/538; G16H 30/20

(52) U.S. Cl. CPC G06N 20/00; G06F 9/54; G16H 30/20; G06F 16/538; G06F 16/258

(57) ABSTRACT

An analysis collaboration platform (ACP) that provides for the connection and management of one or more analysis services with input data and sources of data. The analysis service(s) may be an Artificial Intelligence (AI) service that receives input data, processes the input data, and provides results to a data store or an end-user. In a user-driven process, the ACP receives a request and provides input data to the analysis service. Analysis results are received in a results mode. In a data-driven process, the ACP operates to provide data directly to the analysis service(s), without user participation. In a training mode, the input data is training data, and the training data is generated by the end-user of the service application. The training data may also be incremental training data generated from the analysis results received by the end-user service application.

### CROSS-REFERENCE TO RELATED APPLICATIONS

This application claims priority to U.S. Provisional Patent Application No. 62/587,762, filed Nov. 17, 2017 and U.S. Provisional Patent Application No. 62/590,515, filed Nov. 24, 2017, each entitled “DATA ANALYSIS COLLABORATION ARCHITECTURE AND METHODS OF USE THEREOF.” The disclosure of each application identified above is incorporated herein by reference in its entirety.

### BACKGROUND

Machine learning generally refers to the ability of a computer program to learn without being explicitly programmed, but rather by construction of algorithms that can learn from and make predictions on data. Such predictions or decisions are achieved through building a model from sample inputs or “training data.” The model is applied to new data for making a prediction. Training models may be “unsupervised” or “supervised.” “Supervised” learning problems are used to attempt to predict results where input data can be mapped to a function or discrete output. “Unsupervised” learning allows a model to approach problems with little or no idea as to what the results should look like. An attempt is made to derive structure from data where the effect of the variables is not necessarily known.

Artificial Intelligence (AI) is a term that is broadly used to describe the ability of computers to perform tasks associated with intelligent beings. There are many branches of AI that are directed to, e.g., search, pattern recognition, inference, reasoning, learning, planning, heuristics, genetic programming, etc. AI is also in the early stages of being applied to healthcare technologies and is experiencing rapid adoption. A challenge in applying AI to medical image viewing services for diagnosis or other clinical decisions is to provide training data of sufficient volume, variety, and quality to generate robust and reliable AI algorithms. Furthermore, AI models for medical image viewing typically require supervised learning, therefore they are resource-intensive to create, maintain and update. In particular, expert physicians annotate and/or label the training data, which requires significant manpower, time and costs. As such, a single analysis service or data source may not be sufficiently robust to provide clinically significant analysis.

### SUMMARY

Disclosed herein is an analysis collaboration platform (ACP) that includes at least one analysis integration module that convert requests for the analysis of input data into a request that can be submitted to a respective at least one analysis service and converts results from the analysis service to a standard format for consumption by a user or at least one data source; a collaboration module that communicates to the at least one analysis integration module and receives input data and requests from the end user in a first process; and a gateway module that exposes an API to provide access to the ACP. The ACP provides connection and management of the at least one analysis service, input data, the user, and the at least one data source to receive requests to process the input data by the at least one analysis service and provide results from the at least one analysis service.

In an implementation, an analysis collaboration platform (ACP) is disclosed that includes at least one analysis integration module for providing data format translation and integration with at least one analysis service; a collaboration module for providing communication between a service application and the at least one analysis service; a gateway module that exposes an API to provide at least one input data source for access to the ACP; and a routing and access logic module for routing input data, analysis results, and requests to the ACP. The ACP provides connection and management of the at least one analysis service, the input data, the analysis results, the service application, and the at least one data source in accordance with the requests.

In accordance with an aspect of the present disclosure, a method for analyzing data is described that includes receiving, at an analysis collaboration platform, input data from at least one data source; processing the request or the input data at the analysis collaboration platform to provide the input data to at least one analysis service; receiving, at the analysis collaboration platform, results from the at least one analysis service; and providing the results from the analysis collaboration platform to the at least one data source. The analysis collaboration platform may be operable in a results mode, a training mode, a user-driven mode and/or a data-driven mode. In the results mode, the analysis collaboration platform provides the analysis results to a service application for display to the end-user at a client device. In the training mode, the input data is training data, and the training data is generated by the end-user of the service application. In some implementations, the training data is incremental training data generated from the result data received by the end-user service application. In the user-driven mode, training data is provided to the analysis service by the service application synchronously during a collaboration session.

In accordance with an aspect of the present disclosure, a method for analyzing data is described that includes receiving an input in a user interface of a medical image viewing service application to search for a study stored in a at least one data source; presenting the retrieved study in the user interface of the medical image viewing service application to a user of the medical image viewing service application; initiating a collaboration session from within the user interface to join at least one analysis service as a collaborator with the user of the medical image viewing service application in the collaboration session; receiving, at an analysis collaboration platform, a request from the medical image viewing service application to analyze image data associated with the study, the request being generated in response to the user input in the user interface of the medical image viewing service application; processing the request at the analysis collaboration platform to provide the image data to at least one analysis service in the collaboration session; receiving, at the analysis collaboration platform, results from the at least one analysis service in the collaboration session; providing the results to the medical image viewing service application from the analysis collaboration platform in real time; and presenting the results in the user interface of the medical image viewing service application.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example architecture of an analysis collaboration platform that integrates data sources with analysis services and provides an interactive end user interface;

FIG. 2 illustrates details of the analysis collaboration platform;

FIG. 3 illustrates an example architecture for integrating the analysis services with a service application;

FIG. 4 illustrates the architecture of FIG. 3 having a data integration module;

FIG. 5 illustrates the architecture of FIG. 4 having additional remote access components;

FIG. 6 illustrates an example architecture that is specific to searching, retrieving, viewing and analysis of medical images operating in the data-driven process;

FIG. 7 illustrates an example architecture that is specific to searching, retrieving, viewing and analysis of medical images operating in the user-driven process and also aspects of the data-driven process;

FIGS. 8A-8C illustrate example information flows between architecture components in the user-driven process;

FIGS. 9-11 illustrate example user interfaces associated with a service application in the user-driven results process;

FIGS. 12A and 12B illustrate example information flows between architecture components in the data-driven process;

FIG. 13 illustrates an example user interface associated with a medical image viewing service application in the data-driven process; and

FIG. 14 illustrates an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms.

Overview

The present disclosure provides for an analysis collaboration platform (ACP) for the connection and management of one or more data analysis services (hereafter “analysis services”) with input data, as well for the connection and management of users of the data with analysis services in real time or asynchronously. The ACP acts as an integration point, hub or “data broker” that provides for selective interaction between data, users and analysis services. The analysis service may be an Artificial Intelligence (AI) service that receives input data, processes the input data, and provides results to a data store or an end-user, as well as receives human-generated input labeled data for training, testing, and validation of AI algorithms. For example, one or more analysis services may collaboratively interact with a user of a medical image viewing service application, such as RESOLUTIONMD where an algorithm is being used clinically to make a decision or is being trained with labeled data generated by experts using the medical image viewing application.

The ACP further enables several processes and modes of operation, such as a user-driven process and a data-driven process. Within each process there may be a training mode and a results mode.

In the user-driven process, the ACP receives an explicit request from a user of a service application to access the analysis service (e.g., an Al analysis service) while viewing or interacting with the data. User inputs are provided from the native service application user interface to the analysis service(s) for a seamless, real-time interaction for requesting and receiving analysis results (“results mode”) or for providing labeled data or validation to AI models (“training mode”).

More particularly, in the user-driven results mode, as the user interacts with the service application, one or more analysis services may be invited to collaborate with the user in a session. Where multiple analysis services are available, the service(s) may be selected by the user or routing and access rules may be implemented in the ACP to automatically match the data with an analysis service. As the collaborative interaction proceeds, the ACP provides data (e.g., raw image data) the one or more analysis services, each of which analyzes the data to produce a result. The results are returned to the ACP and subsequently to the service application for viewing. The results (e.g., labeled image data) may also be returned to the data source. The service application may provide for data validation, filtering, and other steps to make the data suitable for processing by the analysis service(s).

In the user-driven supervised training context, the user-driven process may also be used to provide labeled training data as part of the user workflow when collaboratively connected to the ACP, e.g. a physician annotating a medical image to provide a disease diagnosis. This may be the so-called “training mode” or “learning mode”, where the analysis service is collecting data, applying segmentation tools, providing/refining algorithms and getting clinical opinions. Because the ACP provides an integration point for multiple users and analysis services, the ACP may also facilitate consensus or crowdsourcing expert validation of AI algorithm results.

In a data-driven process, the ACP operates to provide data directly to the analysis service(s), without user participation. More particularly, in the data-driven results mode the ACP may apply routing and access rules to determine if data appearing at a data source is appropriate for analysis, and if so, the data is uploaded to the ACP and subsequently to one or more AI services for analysis. Results are sent back to the ACP, the data source, or another location and made available to the user for later viewing, for example for a physician to review an AI diagnosis from within a medical image viewing service application. The data-driven process may also provide data to analysis services for unsupervised training, i.e., data-driven training mode.

Other features of the ACP provide end-to-end management of the functions and processes that occur between analysis vendors, such as Al vendors, data providers, and users, for example, licensing, access to data or analysis services, notifications, billing, analytics, auditing, etc.

Example Architectures

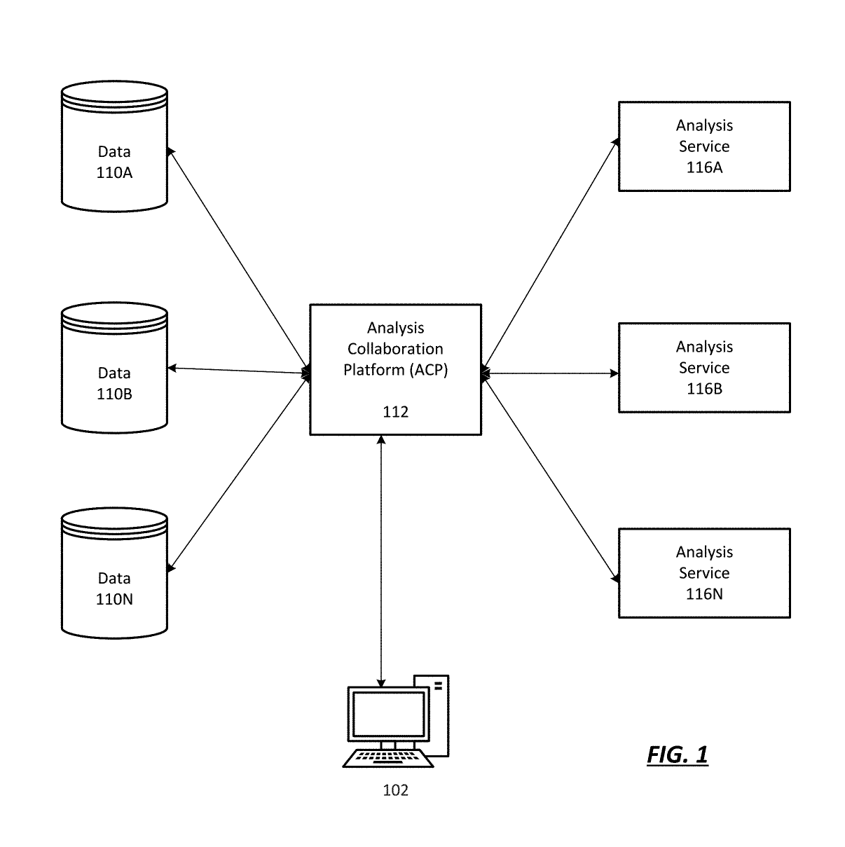

With reference to FIG. 1, there is illustrated an example architecture in which aspects of the present disclosure may be implemented. As illustrated in FIG. 1, an analysis collaboration platform (ACP) 112 may act as a configurable integration point to receive data from multiple data sources 110A/110B/110N and to provide the data to multiple analysis services 116A/116B/116N, and to return results from the multiple analysis services 116A/116B/116N to the multiple data sources 110/110B/110N or other location. The ACP (112) may also be connected to a client (102) connected to a service application, for example, a desktop/notebook personal computer or a wireless handheld device, such as an IPHONE, an ANDROID-based device, a tablet device, etc. In some implementations, plural clients may be connected to the service application. The client 102 (or clients) may request and view data from one or more of the multiple data sources 110A/110B/110N, interact with the data, and submit the data for analysis by one or more of the multiple analysis service 116A/116B/116N. Results are returned from the multiple analysis services 116A/116B/116N to the ACP 112, which may provide the results to the client 102 and/or the multiple data sources 110A/110B/110N. Alternatively, labeled data created by expert users of the service application may be sent to one or more analysis service 116A/116B/116N for training purposes. As such, the ACP 112 is a universal “vendor neutral” integration point that alleviates the IT burden on analysis services users and analysis services vendors who otherwise would need to integrate every analysis service individually into the users' systems.

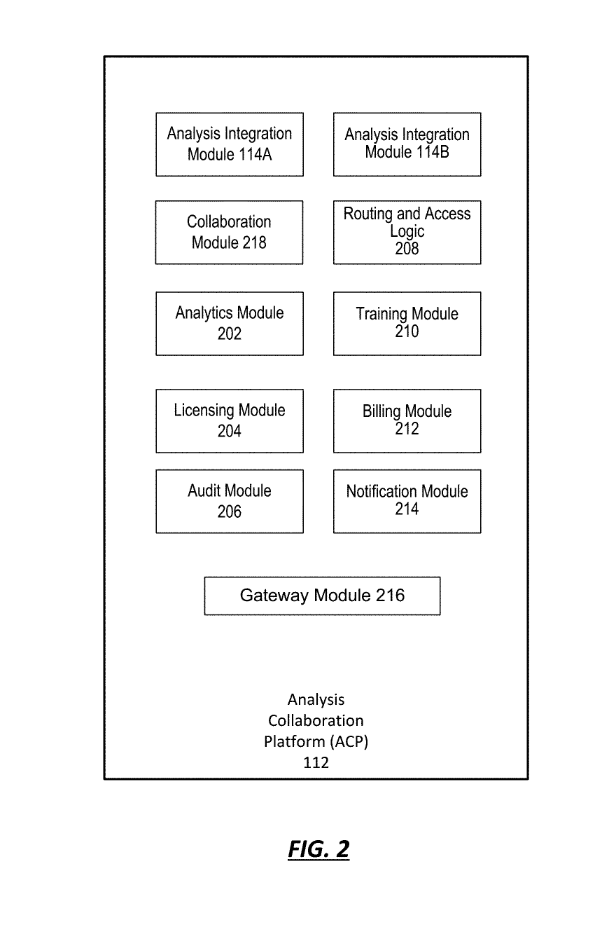

With reference to FIG. 2, there is illustrated example modules that operate within the ACP 112 to provide functionalities and services.

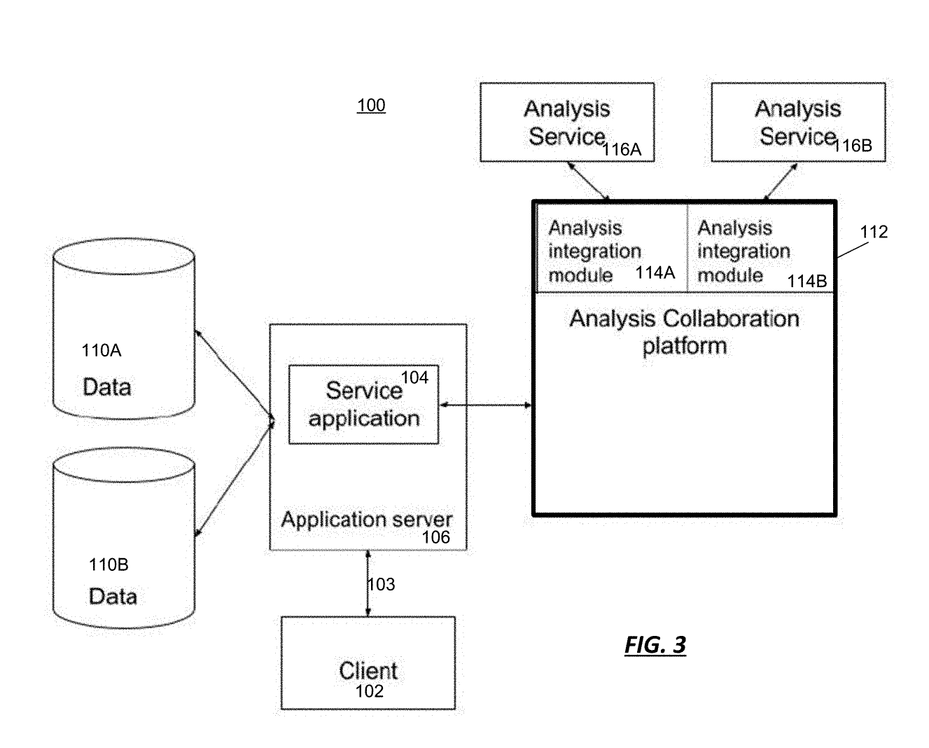

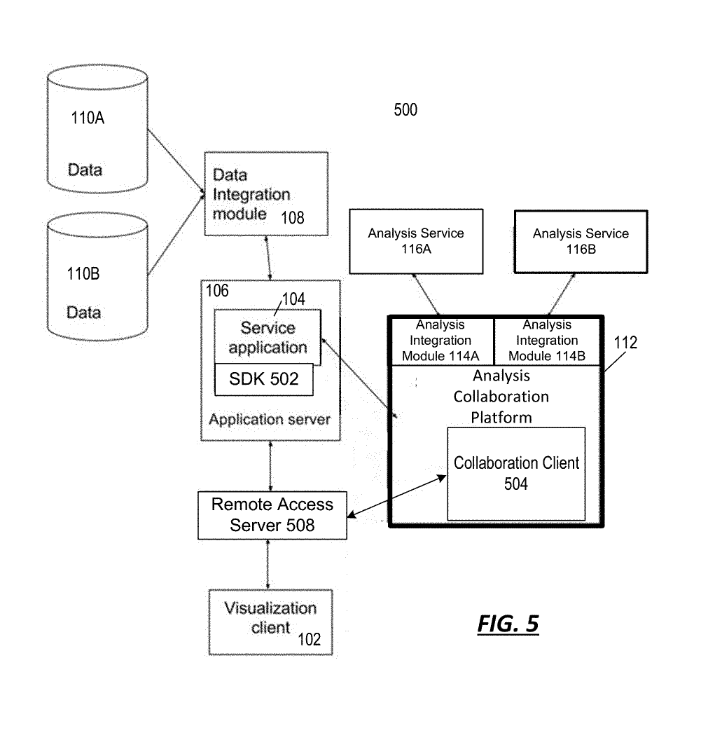

Analysis integration module(s) 114A/114B convert or translate requests for the analysis of some data into a request that can be submitted to an analysis service. The analysis integration module(s) 114A/114B may also convert or translate results from the analysis service to a standard format for consumption by an end user or storage in one of the multiple data sources. More details of the analysis integration module(s) 114A/114B is provided with reference to FIG. 3. A collaboration module 218 provides a collaboration functionality whereby the client 102, interacting with a service application, may join one or more of the multiple analysis service 116A/116B/116N in a collaboration session in the user-driven mode. The collaboration module 218 communicates to the analysis integration module(s) 114A/114B. In a specific implementation, the collaboration module 218 may include a collaboration client 5504 that may, for example, include a client SDK (not shown) that is adapted to receive the input data from a remote access server to which it is connected. More details of the collaboration client 504 are provided with reference to FIG. 5.

An analytics module 202 may keep track of data storage, error rates related to API usage (e.g., API 602, described below), concurrent user log in, how long each AI vendor takes to process, error rates on APIs, etc. Data generated by the analytics module 202 may be used for reporting and usage statistics. The data may also be provided to the billing module 212 for billing purposes.

A licensing module 204 may keep track of which analysis services are available in response to a request received at the ACP 112. For example, a hospital may license one or two analysis services to analyze medical image data. Using information from the licensing module 204, the ACP 112 would route analysis requests to the licensed analysis services in response to a request from the hospital user or data source.

Audit module 206 tracks operations performed by the ACP 112 and provides for traceability of data flows and results.

Routing and access logic 208 provides for AI service selection in accordance with, e.g., input data parameters, a type of study, user selection, information from the licensing module 204, etc. The routing and access logic 208 may provide rules for matching data to analysis services 116A/116B/116N, for example, according to IT or user preconfigured service selection, or dynamic analysis service selection based on availability (e.g., emergencies), subscriptions, or prioritization (e.g., user preferences, preconfigured tiered preferences). Routing and access logic 208 may also provide for rules to automatically generate a second opinion from another algorithm in accordance with threshold confidence levels.

A training module 210 may be provided to communicate training data, including validation data, to the analysis services in accordance with licensing agreements and routing and access rules. The training module 210 may provide for data quality tracking, user-specific custom training, expert validation crowdsourcing of analysis results, user registration and credential validation, quality rankings, certifications, access to a network of verified experts who can produce training data, etc. The training module 210 may allocate and provide storage for training data which can also function as a training data marketplace. For example, a marketplace for expertly-annotated and anonymized medical image data may be useful for organizations requiring large datasets for research purposes. The training module may communicate with reporting systems, for example the analysis service can partially pre-fill a report to help a user more quickly complete the work item. If the user alters the pre-filled report, that action can be taken as an indication that the result was not sufficiently accurate and can be used as a source of training data, or at least a signal that training data should be generated from that study. The report may provide enough labeling, but perhaps additional labelling is required by another expert.

A billing module 212 may be provided to assess costs to users of analysis services. The billing module 221 may also assess costs to the analysis services for training data submitted by the connected users, as well as for purchases of training data from a training data storage market.

A notification module 214 may provide alerts (e.g., positive results in data-driven mode) and notifications relate to a data ingestion API that is made available by the ACP 112.

A gateway module 216 may expose the API 602 to provide access to the ACP functionalities and upstream analysis services.

The ACP 112 may further provide a dashboard and the saving and setting of preferences.

With reference to FIG. 3, there is illustrated an example architecture 300 in which aspects of the present disclosure may be implemented. As illustrated in FIG. 3, the client 102 may be connected by a communication network 103 to an application server 106 running, e.g., a service application 104 such as a medical image viewing service application. The communication connection 103 may be a TCP/IP communications network, a VPN connection, a dedicated connection, etc. Herein, the service application 104 may be any remotely accessed application that provides a functionality in a native user interface to collaboratively engage one or more data analysis services 116A/116B, such as an AI analysis service. The service application 104 may also be considered a “data source” as the service application 104 may retrieve and forward data from the multiple data sources 110A/110B, create its own data (e.g., rendered images), and/or provide training/feedback data.

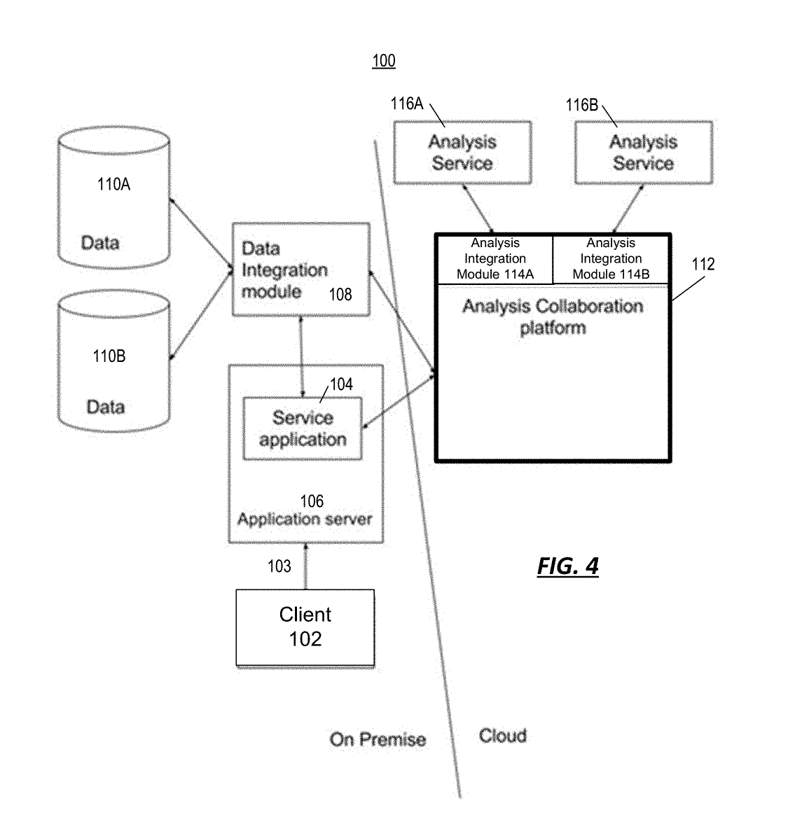

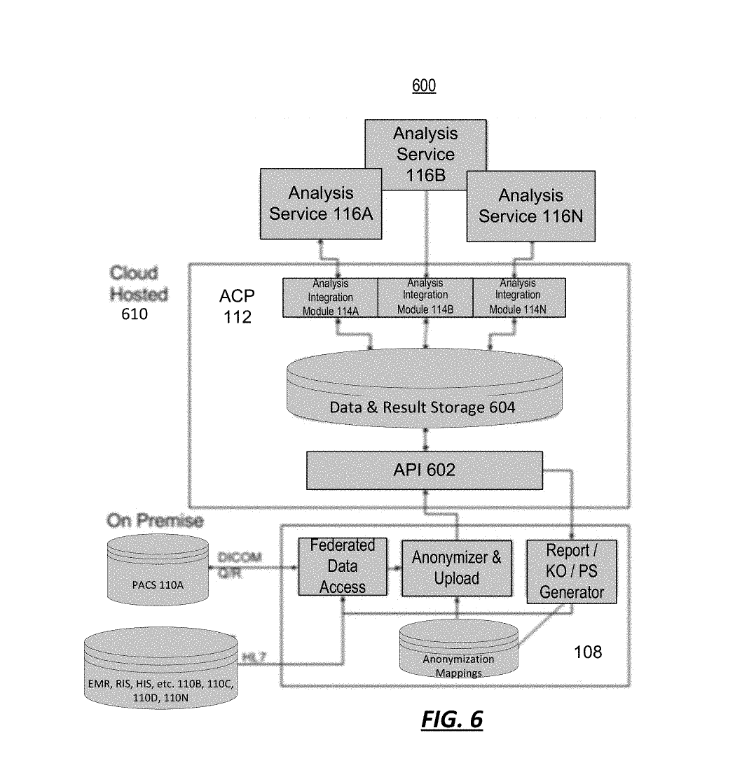

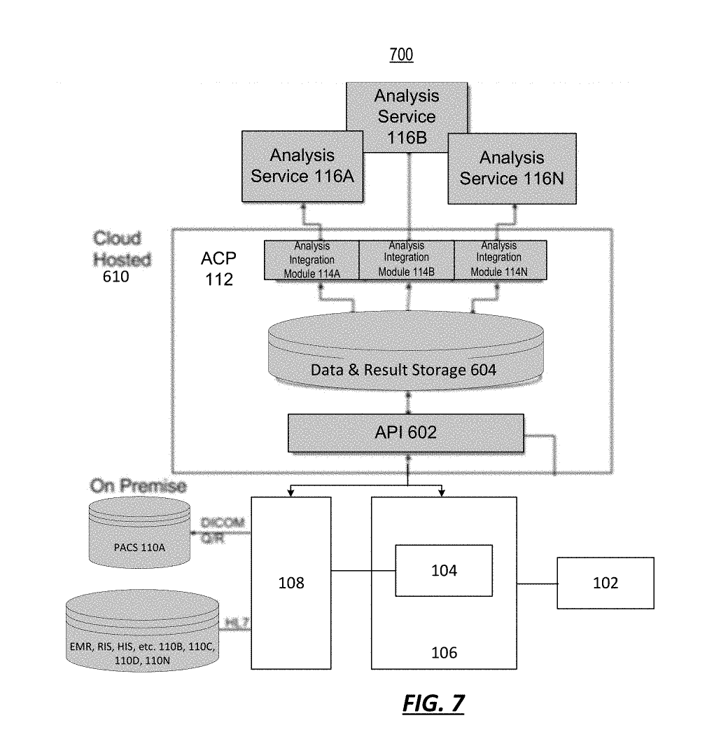

As described above, the ACP 112 may act as an integration point or “data broker” to multiple analysis services 116A/116B that allows for multiple analysis services to access the multiple data sources 110A/110B. As shown in further detail in FIGS. 4-7, the ACP 112 may operate by receiving data from the service application 104 or a data integration module 108 (see, FIG. 4) and providing the data as it is received to an appropriate analysis services 116A/116B/116N for processing. Alternatively, as shown in FIGS. 6-7, the data may be uploaded to the ACP 112 and temporarily stored in, e.g., storage 604 until it can be processed by the appropriate analysis services 116A/116B/116N. In either scenario, the ACP 112 may immediately return the results to the source (i.e., the service application 104 or the data integration module 108) or store the results until they can be returned.

In addition to the functionality described with reference to FIG. 2, the analysis integration module(s) 114A/114B may provide an HTTP API which can be used to submit requests and retrieve results. However, most analysis services will have a slightly different API, so each analysis integration module 114A/114B will contain an amount of custom code to connect the ACP 112 with its respective the analysis application. The analysis service results might be in an arbitrary format defined by that analysis service vendor, as such the analysis integration module 114A/114B may convert that result from a non-standard format to one of a more standardized set of formats, such as, in the case of medical imaging data to DICOM presentation state. The architecture 100 may use an HTTP REST style API. In this example, the ACP 112 loads data directly from the data sources 110A/110B (e.g., a PACS or VNA).

With reference to FIG. 4, the architecture 100 may optionally include the data integration module 108 for bringing data from multiple different data sources 110A/110B together into a common data model or format for ingestion by the ACP 112. The data integration module 108 handles HTTP requests from e.g., the service application 104, to perform a search using user-specified criteria, or to load user-specified data. The data integration module 108 converts the request into whatever protocol is required to communicate to the connected data sources 110A/110B, such as DICOM Q/R, DICOM web, XDS-I, or a propriety API. Once results are retrieved, the data integration module 108 may convert them to a common data format such as JSON or XML for search results, binary data for documents and DICOM objects (no conversion basically) and returns them as an HTTP response. The data integration module 108 provides connections to a variety of data sources 110A/110B by hiding the details of integrating with those data sources 110A/110B and presenting a single interface for information exchange to all of them. The data integration module 108 provides a data path that by-passes the service application 104 in the data-driven process where the ACP 108 operates to provide data directly to the analysis service(s) 116A/116B, without user participation. Also, as shown in FIG. 4, the architecture 100 may be configured in a distributed fashion having on premise components and cloud-based components

With reference to FIG. 5, there is illustrated the example architecture 500 that includes additional remote access components. In the architecture 500, a remote access server 508 may execute on its own physical server or node, or on the application server 106. The remote access server 508 provides features such as managing sessions, marshalling connections from clients, and launching application services. The remote access server 508 manages collaborative sessions, which allows two or more users to view and interact with the same service application(s) 104. The application server 106 may include a server software development kit (SDK) 502 that provides display information to the service application 104 from the client 102. An example of the remote access server 508 is PUREWEB, available from Calgary Scientific, Inc. of Calgary, Alberta, Canada.

As further shown in FIG. 5, the analysis service collaboration client 504 communicates to the analysis integration module(s) 114A/114B. The collaboration client 504 may include a client SDK (not shown) that is adapted to receive the display information from the remote access server 508 to which it is connected. A collaboration notification is provided to initiate a collaboration session between the service application 104 and service collaboration platform 112. In addition, the architecture 500 may be used in conjunction with smartphones, tablets, notebooks or commodity desktops, as the architecture 500 is designed to scale images in accordance with the hardware and display capabilities of the connected client 102.

FIG. 6 illustrates an example architecture 600 that is specific to data-driven searching, retrieving, viewing and analysis of medical images. In the architecture 600, the on-premise components (e.g., the data integration module 108) operate in the data-driven process to anonymize a DICOM study and record a mapping between the original study and the anonymized version. The data integration module 108 may further generate an encryption key whereby original identifiers could be encrypted on premise and uploaded with the anonymized DICOM instances to the cloud-based components. The data integration module 108 may also generate DICOM objects, such as a basic text structure report (SR), key objects (KO), and presentation state (PS), from AI results by linking the anonymized identifiers with the original identifiers. Other specifics of the medical image viewing service application environment in the on-premise components of the architecture 600 are that the data sources 110A/110B may be a PACS, EMR, RIS, HIS, etc. The on-premise components handle integration with client (e.g., hospital) IT systems, data anonymization and linking of AI outputs with original data and patient demographics to create reports specific to the client site. These components may be stateless and may be located in a cloud-based environment. The cloud-hosted components 610 are configured to receive, store and process data by exposing the API 602 that allows the data integration module 108 to access the functionality of the ACP 112. In addition, the ACP 112 may provide for image and results storage 604 to and from the analysis integration modules 114A/114B/114N that interface with the analysis services 116A/116B/116N. The storage of data and results in storage 604 may be multi-tenant such that multiple client facilities (e.g., hospitals) may utilize the ACP 112 to connect to a selected one or more of the 116A/116B/116N. The ACP 112 would further notify an appropriate client facility of the availability of results. The integration modules 114A/114B/114N, in addition to the operations they perform described above, may also opaque identifiers so no patient information is communicated to the analysis services 116A/116B/116N. Although not shown in FIG. 6, the ACP 112 may perform the following tasks, such as, but not limited to receiving a notification when data is available to analyze, retrieving the data to analyze from a cloud storage location, and returning results to the on-premise platform, including version information of the software and model parameters used to compute the result.

FIG. 7 illustrates an example architecture 700 that is specific to user-driven searching, retrieving, viewing and analysis of medical images where the service application 104 executing on the application server 106 is a medical image viewing service application having a collaboration mode whereby one or more of the analysis services 116A/116B/116N may be joined as a collaborator in a session with a user at the client 102. The architecture of FIG. 7 may also be used in the data-driven process. In the architecture 800, the on-premise components operate in the user-driven process. The cloud-hosted components 610 operate as described above with regard to FIG. 6, however, the API 602 that allows both the data integration module 108 and the service application 104 to access the functionality of the ACP 112.

Example Methods of Use

User-Driven Process

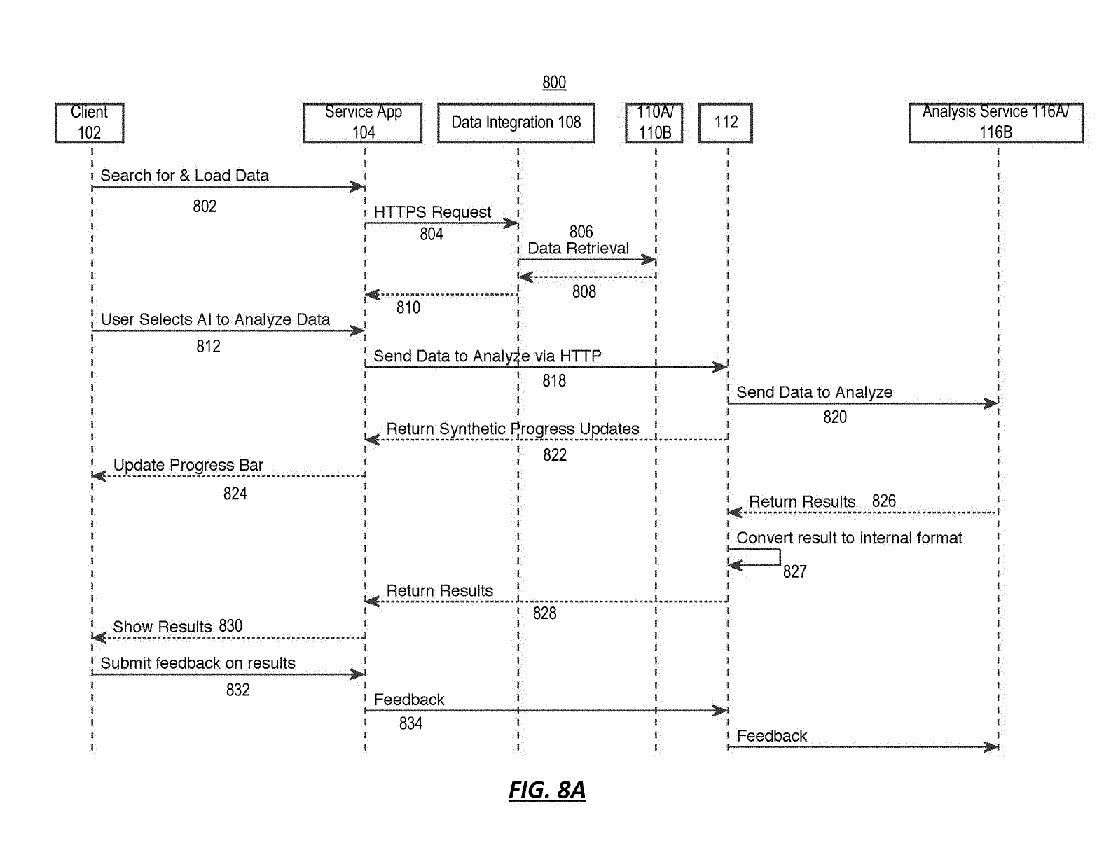

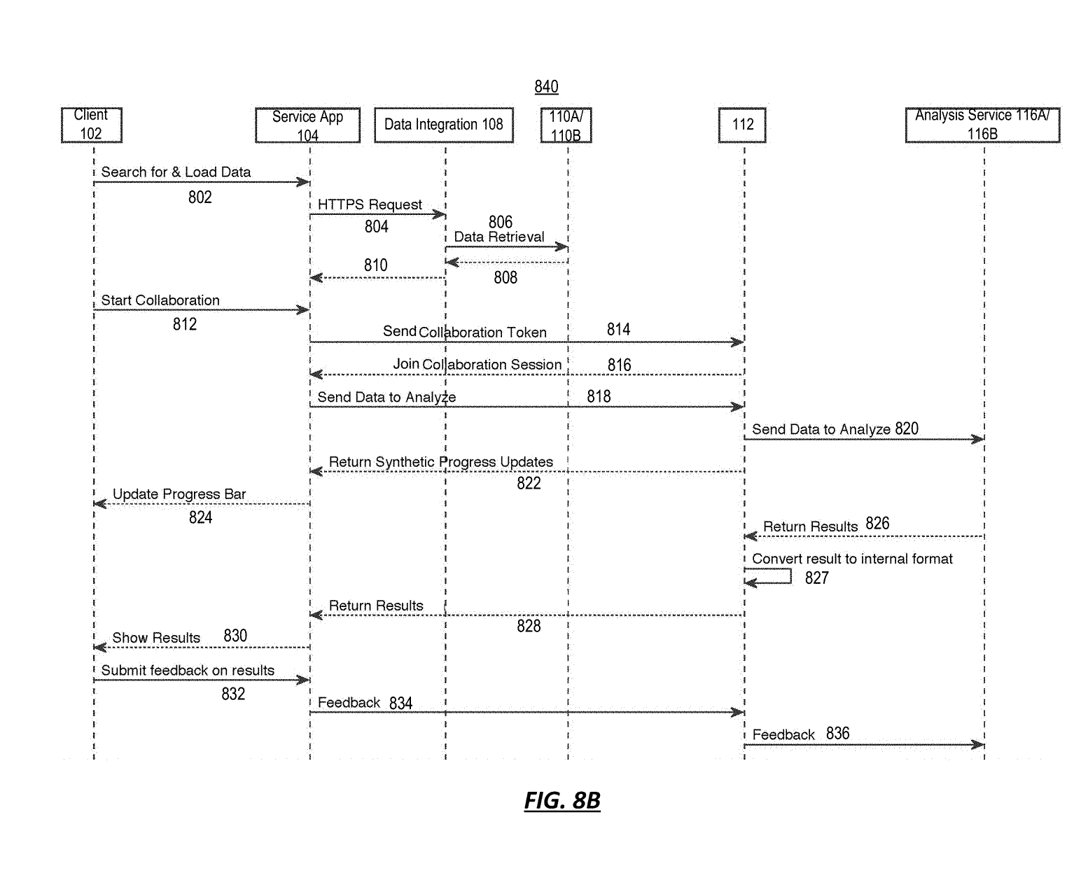

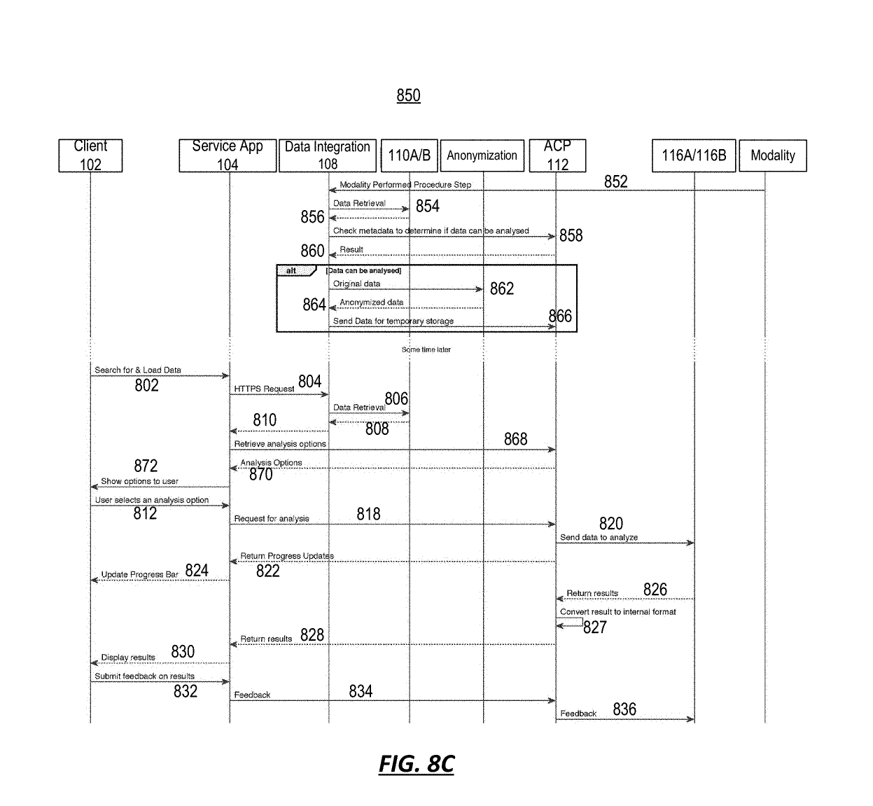

FIG. 8A shows example information flows 800 between architecture components described above in the user-driven process. With reference to FIG. 8A, initially, the user starts the service application 104 at the client 102 and searches for, and loads, a data of interest (flow 802). The service application 104 makes an HTTP request to the data integration module 108 (flow 804), which then accesses the data sources 110A/110B using, e.g., a data retrieval mechanism (flow 806). The data integration module 108 may convert the HTTP requests to an appropriate protocol to search for and retrieve data. Flows 808 and 810 shows data responsive to the search flowing back from the data sources 110A/110B and the integration module 108 to the service application 104. Once the service application 104 has loaded the data, it renders image data that is transmitted to the client 102 for display to the user so the use can view the retrieved data.

During the interaction with the service application 104, a user interface may be presented such that the user may select an AI application(s) 116A/116B to perform analysis using an interface displayed at the client 102 (flow 812). Alternatively, the routing and access logic 208 in the ACP 112 may contain rules that determine which of the AI application(s) 116A/116B are used (e.g., based on contracts, expertise, urgency, etc.). The service application 104 then sends data to be analyzed to the ACP 112 (flow 818). The data communicated at flow 818 may be raw data retrieved from the data sources 110A/110B and/or data created by the service application 104 before flow 818 is executed.

In the case of medical images, the service application 104 may send a full-sized resolution (e.g., original) image to the ACP 112. Next, the ACP 112 sends data to the AI application(s) 116A/116B for analysis (flow 820). The ACP 112 may decide to send the data to one or more of the analysis service 116A/116B in accordance with the type of data received, licensing of the analysis service, routing and access rules, etc. This request to the analysis service 116A/116B may be an HTTP request. The AI application(s) 116A/116B begin analyzing the data and while the AI application(s) 116A/116B is/are computing results, the ACP 112 may produce a synthetic progress update based on, e.g., the average time taken to obtain results from the AI application(s) 116A/116B (flow 822). The client 102 displays a progress bar based on this information. Once the AI application(s) 116A/116B complete their analysis, the results are returned to the ACP 112 (flow 826), optionally converted to an internal format (flow 827) and forwarded to the service application (flow 828). The service application interprets the results and renders them for display to the user at the client 102 (flow 830).

In some implementations, the user may be interacting with the analysis collaboration platform in the training mode. The training mode may be activated or enabled by the routing and access logic 208, whereby routing and access rules may specify that an end-user or entity is authorized to submit training data to the AI application(s) 116A/116B. Once enabled by the routing and access logic 208, the training mode may be activated through a user interface control presented at the client 102. The control provides an indication to the service application 104 that the user is submitting feedback in response to the results provided at flow 828. The feedback may be, e.g., incremental/expert training annotations refining the results that were provided at flow 828. The annotations are communicated from client the service application at flow 832 as input data. The feedback is then communicated from the service application to the ACP (flow 834), which then forwards the feedback to the AI application(s) 116A/116B (flow 836).

In some implementations, the training mode (e.g., flows 832-836) may be performed independently of the other flows shown in FIG. 8A. For example, a user may want to provide training to the Al application(s) 116A/116B regarding results, data, or other information that do not originate from the Al application(s) 116A/116B. As a particular example, the user may be a radiologist analyzing CT or MRI images where the radiologist makes his or her own diagnosis. The radiologist may annotate the images to identify the relevant features of the diagnosis where the annotations are supplied to the AI application(s) 116A/116B as the training data. Other examples would be annotations provided by a seismologist reviewing seismic data, annotations provided by an architect reviewing structural information, etc. The training data may be made available in another data store connected to the ACP that functions as a training data marketplace. The marketplace may operate as a subscription-based service where expertly-annotated and anonymized medical image data is made available for research purposes, etc.

FIG. 8B shows example information flows 840 between architecture components described above in the user-driven process. The information flows 840 in FIG. 8B are similar to the flows 800 in FIG. 8A; however, during the interaction with the service application 104, the user may make a request at the client 102 (flow 812) to start a collaboration session with the Al application(s) 116A/116B. In response, a collaboration token is sent from the service application to the ACP 112 (flow 814). The ACP 112 is joined to the collaboration session (flow 816) and service application 104 sends data to be analyzed to the ACP 112 (flow 818), as described above. As in FIG. 8A, feedback may be provided by a user in a training mode.

In the operational flows of FIG. 8B, the updates shown in flow 822 may be sent to the service application 104, for example, via remote access commands and transmitted to the client 102 via a differencing functionality of an application state model used by the remote access server 508. Details of the application state model may be found in U.S. Pat. No. 8,994,378, which is incorporated herein by reference in its entirety.

FIG. 8C shows example information flows 850 between architecture components described above in the user-driven process with pre-loaded data. Initially, a modality performed procedure using e.g., medical imaging equipment, may produce data to be analyzed. The acquired data may be provided to the data integration module (flow 852), which persistently saves the acquired data to the data sources 110A/110B (flow 854). The data may be retrieved for processing and checks of metadata to determine if the data can be analyzed (flows 856 and 858). The results of the checks are provided to the data integration module (flow 860).

Alternatively or additionally, the data provided by the modality to the data integration module may be communicated to the ACP (flow 862) to be anonymized, as described above. The anonymized data is returned to the data integration module (flow 864) and them may be sent to temporary storage (e.g., storage 604) at the ACP (flow 866). The data is now pre-loaded at the ACP are can be accessed at a later time by a user. The remaining flows of FIG. 8C are similar to those described above in FIG. 8A, including operating in a training mode. The flows of FIG. 8C, however, further add a feature of retrieving analysis options at the service application 104 (flow 868), which are provided by the ACP at flow 870. The options are displayed in a user interface of the client 102 at flow 870, and may include options to select an Al application 116A/116B, provide training data to Al application 116A/116B, etc. A selected analysis option may be included with information provided by the user when an analysis service or collaboration is started at flow 812.

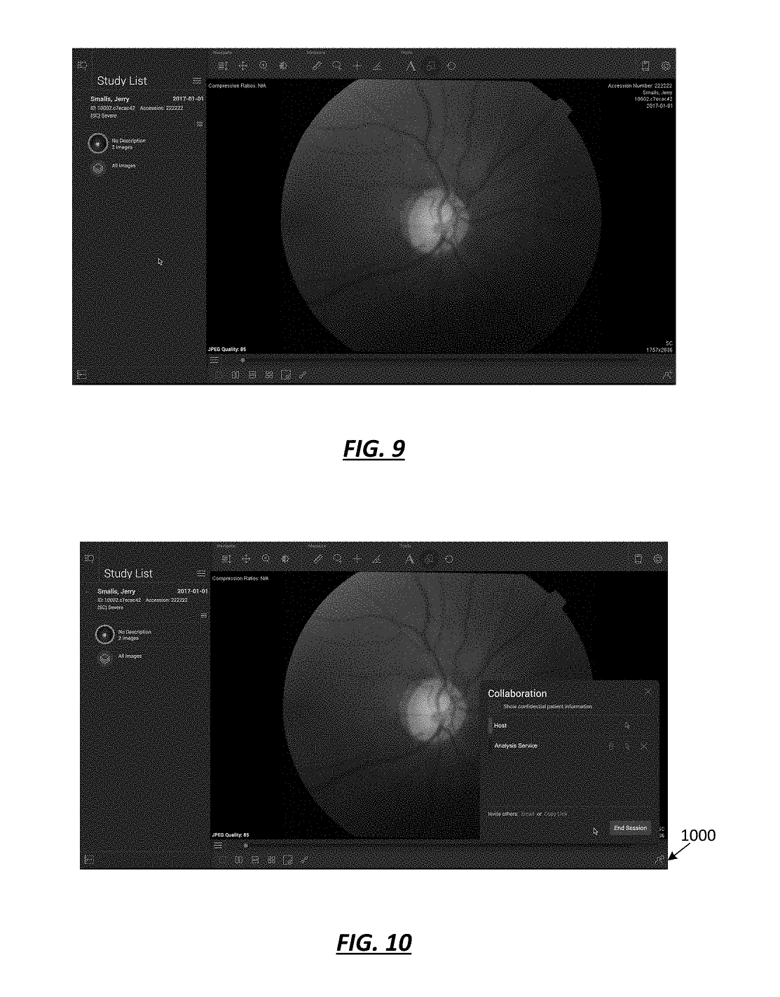

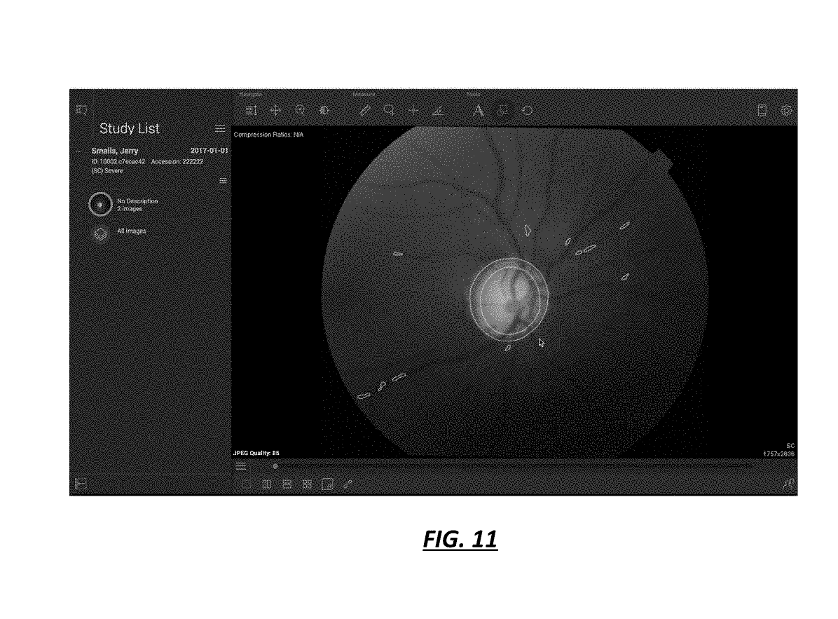

FIGS. 9-11 illustrate example user interfaces that may be presented to a user at the client 102 while interacting with the service application 104 in the user-driven process of operation in the results mode. In the example of FIGS. 9-11, the service application 104 may be a medical image viewing application that is used in the detection and/or diagnosis of diabetic retinopathy. With reference to FIG. 9, a user may select and loads a study for viewing that includes images of an eye. In FIG. 10, the user joins the analysis service as a collaborator by clicking, e.g., a “collaborator” button 1000. The collaboration proceeds as described above where data is provided by the service application to the ACP for analysis by the analysis service. The analysis service performs analysis on the image to identify areas of damage to blood vessels within the eye in real-time. As shown in FIG. 11, the analysis service has performed an analysis on the image to identify the damaged areas. Further analysis may be performed on the image once the areas are identified.

Data-Driven Process

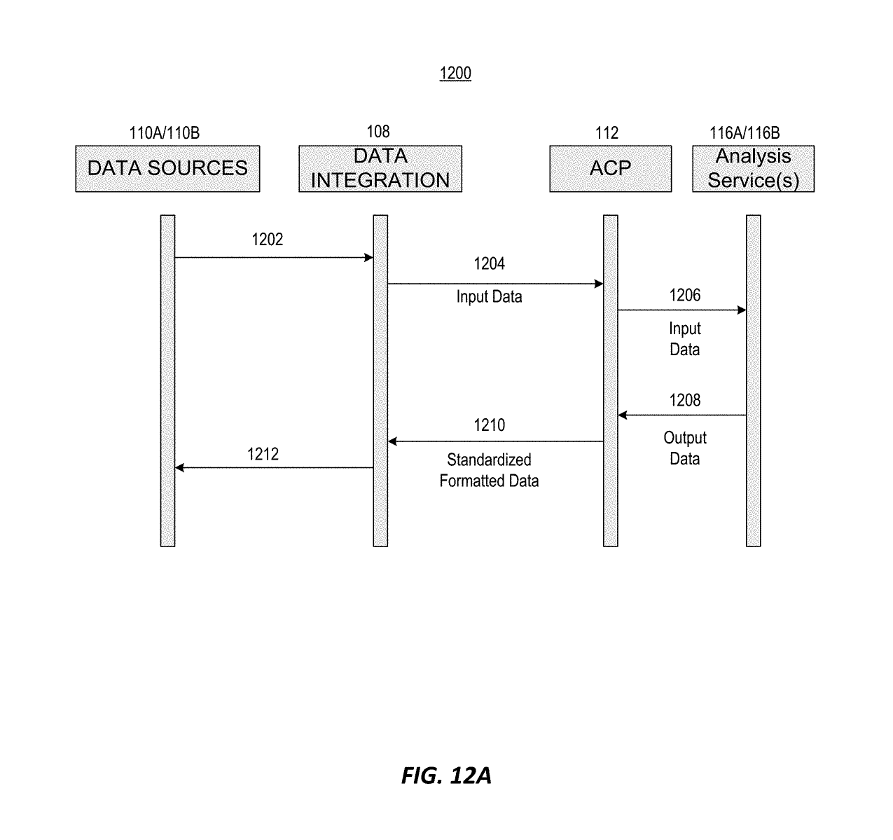

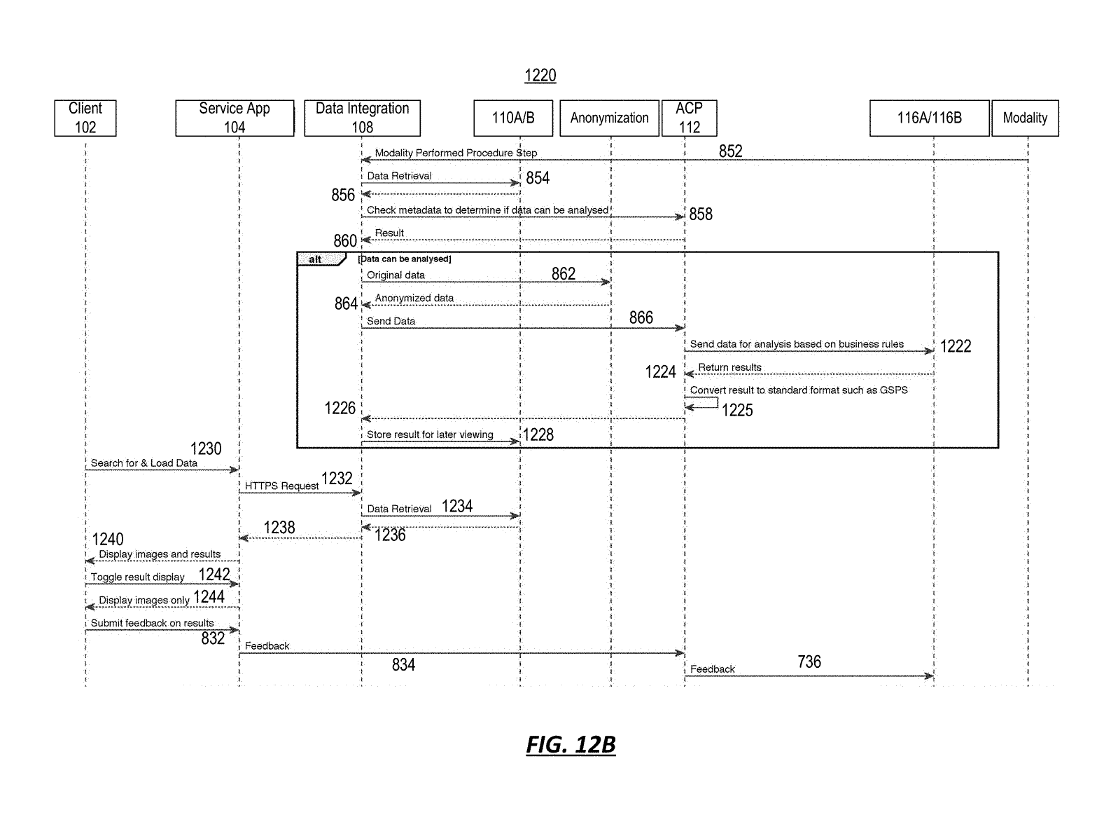

With reference to FIGS. 12A and 12B, there is illustrated a data-driven process, where relevant data may appear or become available on one or more of the data sources 110A/110B/110N for processing. Routing and access rules may be applied to the data to determine if it is ripe for analysis, and if so, it is uploaded to the ACP 112 and communicated to one or more of the analysis services 116A/116B/116N. The results are later returned to the ACP 112 and stored on the appropriate one or more of the data sources 110A/110B/110N, which may be subsequently viewed by a user.

With reference to FIG. 12A, there is a sequence diagram 1200 showing example flows 1200 of a data-driven process. Data on one of the data sources 110A/110B may become available for analysis, as noted above. This data is communicated to the data integration module 108 using, e.g., HTTP/HTTPS request/response flows (flow 1202). The data integration module 108 forwards the data to the ACP 112 (flow 1204), which formats and communicates the data to one or more of the analysis services 116A/116B (flow 1206). For example, the ACP 112 may use HTTP requests to post to requests to the server hosting the analysis services 116A/116B. The ACP 112 may decide to send the data to one or more of the analysis service 116A/116B in accordance with the type of data received, licensing of the analysis service, routing and access rules, etc. When the analysis services 116A/116B has completed the analysis of the data, the output data results are returned to the ACP 112 (flow 1208). The output data may be returned as JSON results. The ACP 112 may then return the results to the data integration module 108 (flow 1210). The results may be repackaged into standardized formatted data for consumption by the data integration module 108. The standardized formatted data is then returned to the data sources 110A/110B for retrieval (flow 1212).

The selection of the one or more analysis services 116A/116B may be based on criteria in addition to those noted above. For example, in a medical image viewing service application context, the selection may be based on a modality or body part scanned. A user may be able to toggle results ON/OFF from each selected analysis service. In the data-driven process, in the medical image viewing service application context, a notification of new studies available for processing may be provided. A scheduled job may run nightly to check for studies of interest or a component may execution an application server that listens for messages indicating that a new study is available.

FIG. 12B illustrates another example sequence diagram 1220 that describes additional details of the modes of operation and collaboration within the architectures of the present disclosure. Flows 852-866 shown in FIG. 12B are performed as described in FIG. 8C, above. After flow 866, the data to be analyzed is communicated to the analysis service(s) 116A/116B according to the routing and access rules in the routing and access logic 208 (flow 1222). After analysis is performed by the analysis service(s) 116A/116B results are returned to the ACP (flow 1224). Optionally, the results are converted to a standard format such as grayscale softcopy presentation state (GSPS) (flow 1225). The ACP then returns results to the data integration module 108 (flow 1226). The data integration module 108 sends the results to the data sources 110A/110B for persistent storage (flow 1228).

Once the results are saved in the data sources 110A/110B, they are available for a user to search, load and review them. For example, user client 102 may search for, and request data to be loaded via the service application 104 (flow 1230). The service application 104 sends the request to the data integration module 108, for example as a HTTPS request (flow 1232). The data integration module 108 then retrieves data from the data sources 110A/110B (flow 1234). The retrieved data is then returned from the data sources 110A/110B to the data integration module 108 (flow 1236), which then returns the data to the service application 104 (flow 1238). The images and results are then displayed at the client 102 (flow 1240). As the user at the client 102 is viewing the images, the results generated by the analysis service(s) 116A/116B may be optionally toggled on and off in accordance with the user control (flow 1242 and 1244). After viewing the results, the that may be submitted in accordance with flows 832-836, as described above with regard to FIG. 8A.

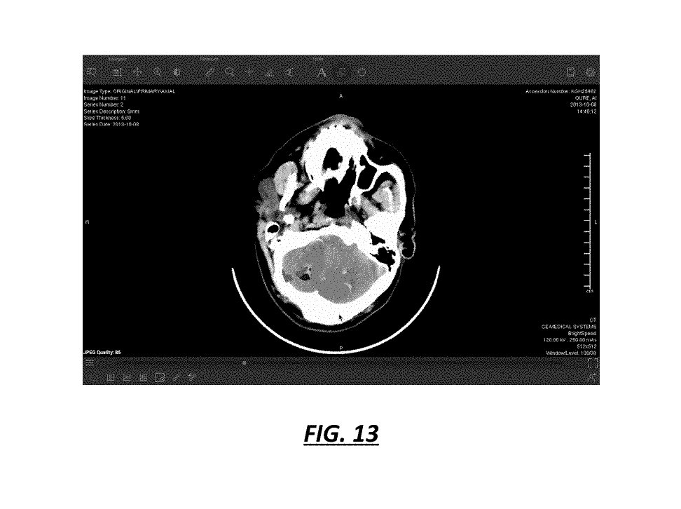

FIG. 13 illustrates an example user interface that may be presented to a user at the client 102 while interacting with the service application 104 in the data-driven process of operation. In the data-driven mode, the results are processed by one or more of the analysis services 116A/116B/116N in accordance with routing and access rules contained in the routing and access logic 208, etc., and the user is notified that the results are ready for viewing. In the example of FIG. 13, the service application 104 may be a medical viewing application that enables a user to search and retrieve a study. In particular, FIG. 13 shows results generated by the one or more of the analysis services 116A/116B/116N showing area of brain hemorrhages in red and orange.

Application to Medical Image Analysis and Interpretation

The present disclosure provides architectures that may be used in the environments where the service application is medical image viewing service application, the data to be analyzed is patient medical image data, and the analysis service is an AI application for diagnosis of medical images. The implementations described herein preserve anonymity of patients and limits the exposure of personal information to the cloud. Further, the architectures may be easily integrated with a wide variety of PACS systems.

With particularity to clinical users, the implementations of the present disclosure provide: (1) real-time interaction with one or more Al services to explore different opinions; (2) offline viewing of results and reports produced by Al services to aid clinical decision making; and (3) AI augmentation of radiologists to help them improve their effectiveness and ability to provide patient care. The architectures described herein provide for storing of the results of an AI analysis in the PACS alongside the original study. This means that the PACS continues to serve as a single source of truth. The architectures also provide an integration point to enable users to leverage a variety of analysis services through a network of analysis service partners. Software components may be provided to aid in the display of AI results and tools to create and submit new training data to AI models. The seamless integration of an analysis with the service application, such as a medical image viewing service application, allows the physician to utilize the AI service without stepping out of the native image service application user interface. In addition to receiving the requested AI results, the implementations also enable a physician to submit labeled training data as part of a normal workflow or tweak returned AI results and submit a new package of training data within the diagnostic workflow, which will serve to refine the training data such that future results generated by the analysis service and more accurate.

For health care institutions, the implementations of the present disclosure insure that analysis service vendors do not have access to original patient data by separating the identifying information from the image in situations where the image is the only data required to make a diagnosis. This makes it more secure and less vulnerable to reportable disclosure under HIPAA. For example, once a study is available in the on-premise PACS, it is uploaded to the ACP in the cloud and anonymization is reversed when results are returned to the PACS and stored as a new series within the original study. The implementations herein also provide a platform whereby hospitals can create a print-ready DICOM reports from the analysis, which conventionally is an undertaking in itself. The report may be customizable for both the hospital and the analysis service vendor. In particular, keeping report generation on premise reduces the risk of leaking patient confidential information. Integration of results into the medical image viewing service application also enables the provision of notifications as soon as reports, PR, KO, or other types of results are available, or it is automatically available in their work list, etc. The notifications may be directly communicated to interested parties to view the results on the imaging viewer.

As described above, the architectures enable the selection of one or more analysis services, where the selection of an appropriate analysis service may be based on many different, configurable criteria. Access to multiple analysis services for the same type of data, will enable the end user to obtain multiple opinions for the same data (e.g., a second concurrent or subsequent opinion as requested or automated according to confidence levels, for expert or Al validation or credentialization, etc.) or to obtain more than one type of diagnosis for the same data, which results may be combined and returned within the same view. A single on-premise installation further allows access to multiple analysis services without a need to manage tools from each analysis service vendor. Feedback from users can be used to train AI models to customize the AI model for a specific site or user. The present disclosure also provides for efficiencies, as users and support personnel need only be trained for one system. Also, a managed DICOM interface minimizes the impact on PACS systems.

Finally, for the analysis service vendors, the architectures described herein enable vendors to sell their products to a wide variety of customers by easing business relationships and overcoming technical integration hurdles. For example, there is no need to develop an uploader, anonymization, viewer, or to manage billing, or on-premise integration. The architectures provide a mechanism for users to easily provide feedback to the analysis service for incremental training, which provides access to experts to generate training data and improves performance of general models. As such, analysis service vendors may be able to offer specific models trained specifically for a particular site or user. Listening to HL7 messages, loading data from a PACS, decompressing it, applying appropriate transformations, and otherwise getting it ready for processing by an analysis service vendor is a significant amount of work, and the architectures herein reduce the level of effort for the analysis service vendors to obtain data, process the data and return the results to the end users. For example, notification of available studies can be converted from HL7 to web standards that can easily be used by analysis service vendors. The medical image viewing service application itself can be extended to provide enhanced interactive visualization capabilities for in depth exploration of AI results.

Thus, as described herein, present disclosure provides for architectures that easily integrate one or more analysis services to perform analysis of data, such as images. Although specific examples were provided, one of ordinary skill in the art would understand that any analysis may be performed on any type of data to achieve the desired result. For example, the data and processing may include natural language processing, unstructured data, computer vision data, robotics, automated learning and scheduling, audio data, historical data analysis, vehicular traffic analysis, environmental data analysis, etc.

Computing Device

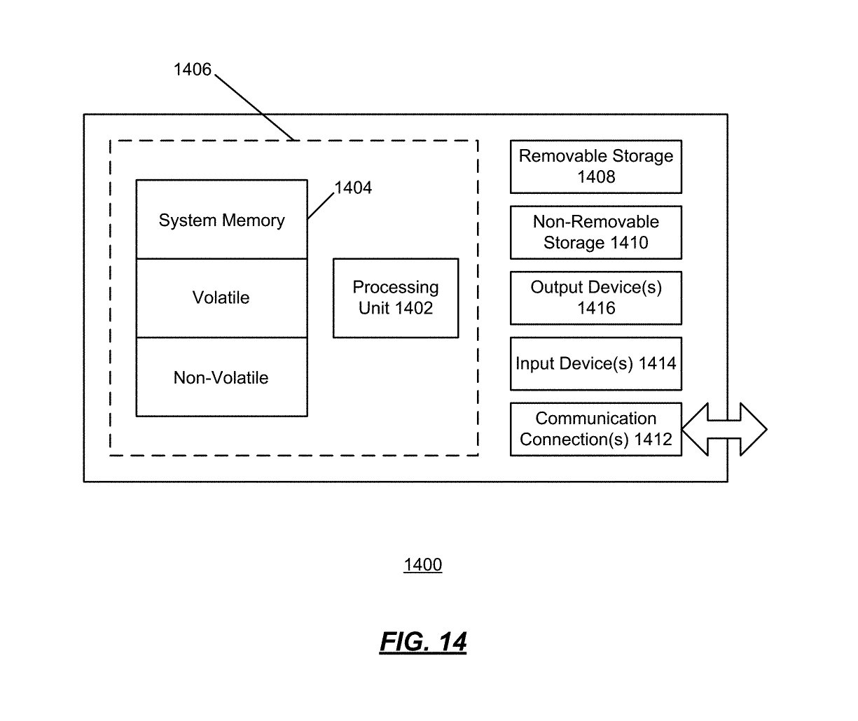

FIG. 14 shows an exemplary computing environment in which example embodiments and aspects may be implemented. The computing system environment is only one example of a suitable computing environment and is not intended to suggest any limitation as to the scope of use or functionality.

Numerous other general purpose or special purpose computing system environments or configurations may be used. Examples of well-known computing systems, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 14, an exemplary system for implementing aspects described herein includes a computing device, such as computing device 1400. In its most basic configuration, computing device 1400 typically includes at least one processing unit 1402 and memory 1404. Depending on the exact configuration and type of computing device, memory 1404 may be volatile (such as random-access memory (RAM)), non-volatile (such as read-only memory (ROM), flash memory, etc.), or some combination of the two. This most basic configuration is illustrated in FIG. 14 by dashed line 1406.

Computing device 1400 may have additional features/functionality. For example, computing device 1400 may include additional storage (removable and/or non-removable) including, but not limited to, magnetic or optical disks or tape. Such additional storage is illustrated in FIG. 14 by removable storage 1408 and non-removable storage 1410.

Computing device 1400 typically includes a variety of tangible computer readable media. Computer readable media can be any available media that can be accessed by device 1400 and includes both volatile and non-volatile media, removable and non-removable media.

Computer storage media include tangible volatile and non-volatile, and removable and non-removable media implemented in any method or technology for storage of information such as computer readable instructions, data structures, program modules or other data. Memory 1404, removable storage 1408, and non-removable storage 1410 are all examples of computer storage media. Computer storage media include, but are not limited to tangible media such as RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1400. Any such computer storage media may be part of computing device 1400.

Computing device 1400 may contain communications connection(s) 1412 that allow the device to communicate with other devices. Computing device 1400 may also have input device(s) 1414 such as a keyboard, mouse, pen, voice input device, touch input device, etc. Output device(s) 1416 such as a display, speakers, printer, etc. may also be included. All these devices are well known in the art and need not be discussed at length here.

It should be understood that the various techniques described herein may be implemented in connection with hardware or software or, where appropriate, with a combination of both. Thus, the methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computer, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device.

One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. An analysis collaboration platform (ACP), comprising:
at least one analysis integration module for providing data format translation and integration with at least one analysis service;
a collaboration module for providing communication between a service application and the at least one analysis service;
a gateway module that exposes an API to provide at least one input data source for access to the ACP; and
a routing and access logic module for routing input data, analysis results, and requests to the ACP,
wherein the ACP provides connection and management of the at least one analysis service, the input data, the analysis results, the service application, and the at least one data source in accordance with the requests.

2. The ACP of claim 1, further comprising a data integration module for converting input data retrieved from the at least one data source and converted into a common format for ingestion by the ACP.

3. The ACP of claim 1, wherein the ACP selectively communicates with plural data sources and plural analysis services in accordance with information contained in the routing and access logic module.

4. The ACP of claim 1, further comprising temporary data storage for storing the input data and the analysis results.

5. The ACP of claim 1, wherein the service application is joined as a collaborator in a collaborative session.

6. The ACP of claim 1, wherein the ACP is operable in a user-driven mode or a data-driven mode.

7. The ACP of claim 7, wherein in the results mode, the ACP provides the analysis results to the service application for display to the end-user at a client device.

8. The ACP of claim 7, wherein the ACP operates in the user-driven mode during a collaboration session, and wherein the ACP provides the analysis results to the end-user synchronously.

9. The ACP of claim 6, wherein in the data-driven mode, the ACP receives the input data from the at least one data source, and wherein the input data is processed asynchronously by the at least one analysis service.

10. The ACP of claim 9, wherein the data integration module provides a data path for the input data to be received by the ACP without use of the service application.

11. The ACP of claim 1, wherein the ACP is operable in a results mode or a training mode.

12. The ACP of claim 11, wherein in the training mode, the input data is training data, and the training data is generated by the end-user of the service application.

13. The ACP of claim 12, wherein the training data is incremental training data generated from the result data received by the end-user service application.

14. The ACP of claim 12, wherein in the user-driven mode, training data is provided to the analysis service by the service application synchronously during a collaboration session.

15. The ACP of claim 12, wherein the training data is provided to a data store.

16. The ACP of claim 1, wherein the mode of operation is determined by, information contained in the routing and access logic module to forward the input data to the at least one analysis service.

17. A method for analyzing data, comprising:
receiving, at an analysis collaboration platform, input data from at least one data source;
processing the request or the input data at the analysis collaboration platform to provide the input data to at least one analysis service;
receiving, at the analysis collaboration platform, results from the at least one analysis service; and
providing the results from the analysis collaboration platform to the at least one data source.

18. The method of claim 17, further comprising joining the at least one analysis service as a collaborator with a user of a service application using a collaboration module at the analysis collaboration platform.

19. The method of claim 18, further comprising receiving feedback from the service application in response to the results; and providing the feedback to the at least one analysis service to refine the results.

20. The method of claim 17, further comprising providing the input data to at least one analysis service in accordance with information contained in a routing and access logic module within the analysis collaboration platform.

21. The method of claim 17, further comprising selectively providing the data to plural analysis services in response to second information contained in the provide the input data to at least one analysis service.

22. The method of claim 17, further comprising:
operating the analysis collaboration platform in a training mode; and
providing the input data in the form of training data that is generated at a service application.

23. The method of claim 22, wherein the training data is incremental training data generated from the results that are received at the service application.

24. The method of claim 22, further comprising providing the training data synchronously during a collaboration session.

25. The method of claim 22, further comprising storing the training data in a data store.

26. A method for analyzing data, comprising:
receiving an input in a user interface of a medical image viewing service application to search for a study stored in a at least one data source;
presenting the retrieved study in the user interface of the medical image viewing service application to a user of the medical image viewing service application;
initiating a collaboration session from within the user interface to join at least one analysis service as a collaborator with the user of the medical image viewing service application in the collaboration session;
receiving, at an analysis collaboration platform, a request from the medical image viewing service application to analyze image data associated with the study, the request being generated in response to the user input in the user interface of the medical image viewing service application;
processing the request at the analysis collaboration platform to provide the image data to at least one analysis service in the collaboration session;
receiving, at the analysis collaboration platform, results from the at least one analysis service in the collaboration session;
providing the results to the medical image viewing service application from the analysis collaboration platform in real time; and
presenting the results in the user interface of the medical image viewing service application.
